(19) United States
(12) Patent Application Publication
(10) Pub. No.: US 2019/0099127 A1
(43) Pub. Date: Apr. 4, 2019

(54) FOOD PURCHASE SUPPORTING SYSTEM AND IMAGE FORMING APPARATUS

(71) Applicant: KYOCERA Document Solutions Inc., Osaka (JP)

(72) Inventor: Yukihiro Nakao, Osaka-shi (JP)

(73) Assignee: KYOCERA Document Solutions Inc., Osaka (JP)

(21) Appl. No.: 16/139,257

(22) Filed: Sep. 24, 2018

(30) Foreign Application Priority Data
Sep. 29, 2017 (JP) 2017-189466

(51) Int. Cl. A61B 5/00; A61B 5/02; A61B 5/11; G06F 3/12; G06F 17/30; G16H 20/60

(52) U.S. Cl. CPC A61B 5/681; A61B 5/02; A61B 5/1118; A61B 5/742; G16H 10/65; A61B 5/0022; G06F 17/30867; G16H 20/60; G06F 3/1292

(57) ABSTRACT

A food purchase supporting system includes a wearable terminal device and an image forming apparatus. The wearable terminal device stores a measurement value with respect to an indicator for management of health of a user. The image forming apparatus includes a first communication device, an acquisition section, a determination section, and a generation section. The first communication device communicates with the wearable terminal device. The acquisition section acquires the measurement value from the wearable terminal device via the first communication device. The determination section determines a food ingredient recommended for the user to ingest based on the measurement value and a target value with respect to the indicator for the management of health of the user. The generation section generates a list of food recommended for the user to purchase based on the food ingredient.

### INCORPORATION BY REFERENCE

The present application claims priority under 35 U.S.C. § 119 to Japanese Patent Application No. 2017-189466, filed on Sep. 29, 2017. The contents of this application are incorporated herein by reference in their entirety.

### BACKGROUND

The present disclosure relates to a food purchase supporting system and an image forming apparatus.

One example of a healthcare information providing system includes a management device, an information providing device, and a user terminal. The information providing device acquires a physical examination result from the management device and determines or calculates a deficient nutritional component and caloric need based on the physical examination result. The information providing device selects ingredients and dishes to recommend based on the deficient nutritional component and the caloric need, and displays the recommendations on the user terminal.

### SUMMARY

A food purchase supporting system according to an aspect of the present disclosure includes a wearable terminal device and an image forming apparatus communicatively connected to the wearable terminal device. The wearable terminal device is for a user to wear on a body thereof. The wearable terminal device stores a measurement value with respect to an indicator for management of health of the user. The image forming apparatus includes a communication device, an acquisition section, a determination section, and a generation section. The communication device communicates with the wearable terminal device. The acquisition section acquires the measurement value from the wearable terminal device via the communication device. The determination section determines a food ingredient recommended for the user to ingest based on the measurement value and a target value with respect to the indicator. The generation section generates a list of food recommended for the user to purchase based on the food ingredient.

An image forming apparatus according to another aspect of the present disclosure is communicatively connected to a wearable terminal device. The wearable terminal device is for a user to wear on a body thereof. The image forming apparatus includes a communication device, an acquisition section, a determination section, and a generation section. The communication device communicates with the wearable terminal device. The acquisition section acquires a measurement value with respect to an indicator for management of health of the user from the wearable terminal device. The determination section determines a food ingredient recommended for the user to ingest based on the measurement value and a target value with respect to the indicator. The generation section generates a list of food recommended for the user to purchase based on the food ingredient.

### BRIEF DESCRIPTION OF THE DRAWINGS

FIG. 1 is a diagram illustrating connection between an image forming apparatus, a wearable terminal device, a point of sale (POS) terminal device, and a server according to an embodiment of the present disclosure.

FIG. 2 is a diagram illustrating a configuration of the image forming apparatus according to the embodiment of the present disclosure.

FIG. 3 is a diagram illustrating a configuration of the wearable terminal device according to the embodiment of the present disclosure.

FIG. 4 is a diagram illustrating a configuration of a first controller and a configuration of a second controller.

FIG. 5 is a screen image illustrating an example of a food list display screen.

FIG. 6 is a diagram illustrating an example of a grocery shelving unit.

FIG. 7 is a flowchart illustrating an example of a process performed by the first controller.

FIG. 8 is a flowchart illustrating the example of the process performed by the first controller.

FIG. 9 is a flowchart illustrating an example of a process performed by the second controller.

FIG. 10 is a flowchart illustrating the example of the process performed by the second controller.

### DETAILED DESCRIPTION

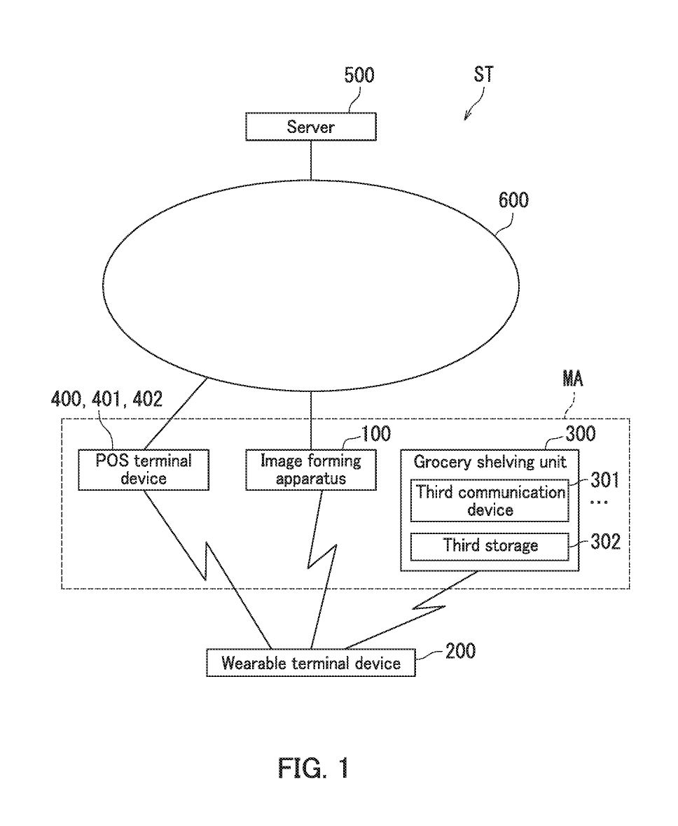

The following describes an embodiment of the present disclosure with reference to the drawings (FIGS. 1 to 10). Note that in the drawings, elements that are the same or equivalent are labelled using the same reference signs and description thereof is not repeated.

First, connection between an image forming apparatus 100, a wearable terminal device 200, a point of sale (POS) terminal device 400, and a server 500 according to the embodiment of the present disclosure will be described with reference to FIG. 1. As illustrated in FIG. 1, the image forming apparatus 100 is communicatively connected to the POS terminal device 400 and the server 500 via a network 600.

The image forming apparatus 100 is also communicatively connected to the wearable terminal device 200 through short-range wireless communication. The POS terminal device 400 is communicatively connected to the wearable terminal device 200 through short-range wireless communication. The term “short-range wireless communication” means wireless communication within a short communication distance (for example, 1 m), and examples thereof include BLUETOOTH (registered Japanese trademark).

The image forming apparatus 100 and the POS terminal device 400 are installed in a store MA. The store MA has a grocery shelving unit 300. Items of food for purchase by users are placed on the grocery shelving unit 300.

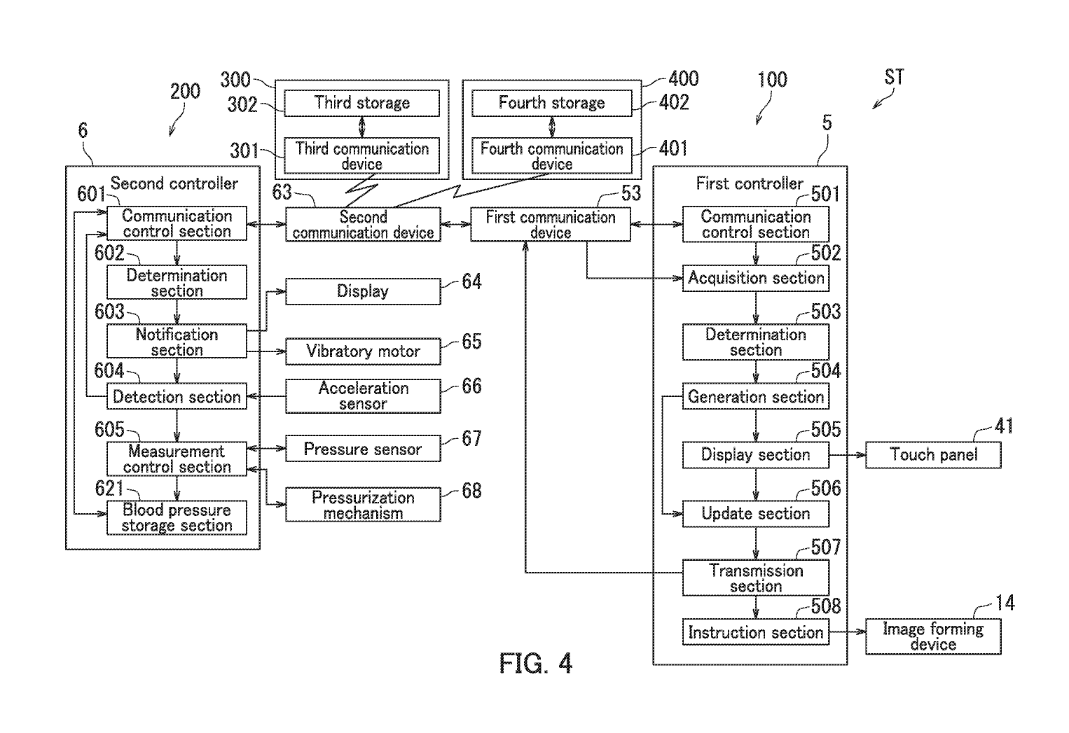

The image forming apparatus 100 is a so-called multifunction peripheral and has a communication function. The image forming apparatus 100 exchanges various information with the wearable terminal device 200 through short-range wireless communication. The image forming apparatus 100 also acquires information indicating a target value HT from the server 500 via the network 600. The target value HT means a value to be achieved with respect to an indicator for management of health of a user. The target value HT is for example a blood pressure level. The target value HT will be described in detail with reference to FIG. 4.

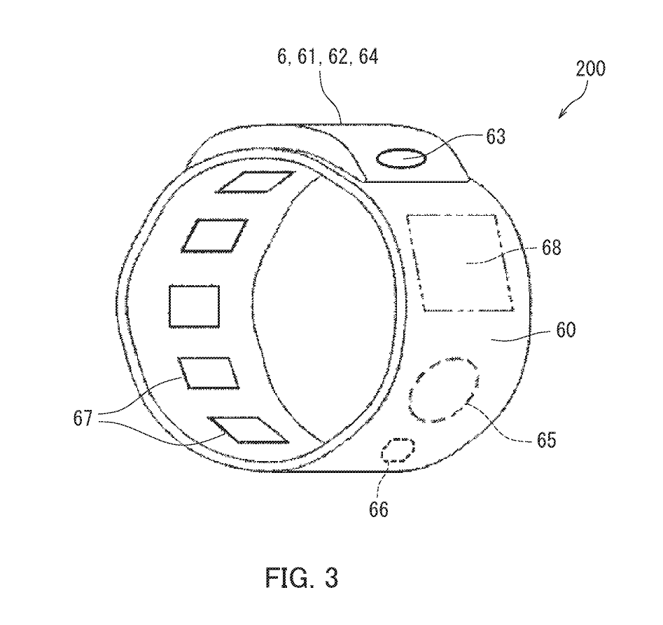

The wearable terminal device 200 is for the user to wear and has a short-range wireless communication function. The wearable terminal device 200 stores information indicating a measurement value HM. The measurement value HM means a value measured with respect to the indicator for management of health of the user. A configuration of the wearable terminal device 200 will be described in detail with reference to FIG. 3.

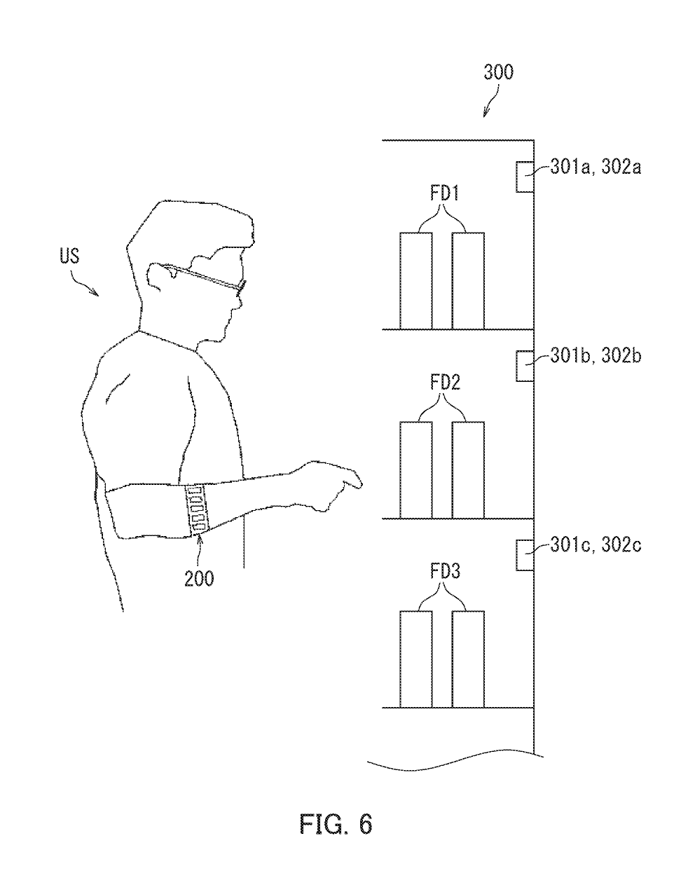

A third communication device 301 and third storage 302 are provided on the grocery shelving unit 300. The third communication device 301 is communicatively connected to the wearable terminal device 200 through short-range wireless communication. The third storage 302 stores therein food information FJ indicating types of the food on the grocery shelving unit 300. A configuration of the grocery shelving unit 300 will be described in detail with reference to FIG. 6.

The POS terminal device 400 aggregates sales figures resulting from selling goods (food, according to the embodiment of the present disclosure) on a product-by-product basis. The POS terminal device 400 has a communication function. The POS terminal device 400 is communicatively connected to a server (not illustrated) via the network 600. The POS terminal device 400 also has a short-range wireless communication function. Specifically, the POS terminal device 400 includes a fourth communication device 401 and fourth storage 402. The fourth communication device 401 is communicatively connected to the wearable terminal device 200 through short-range wireless communication.

The server 500 has a communication function. The server 500 stores information indicating the target value HT. The target value HT is for example determined based on a physical examination. The target value HT may alternatively be determined based on age and gender.

The network 600 is for example the Internet. Note that the network 600 is not limited to the Internet. The network 600 may be a wide area network (WAN) or a local area network (LAN).

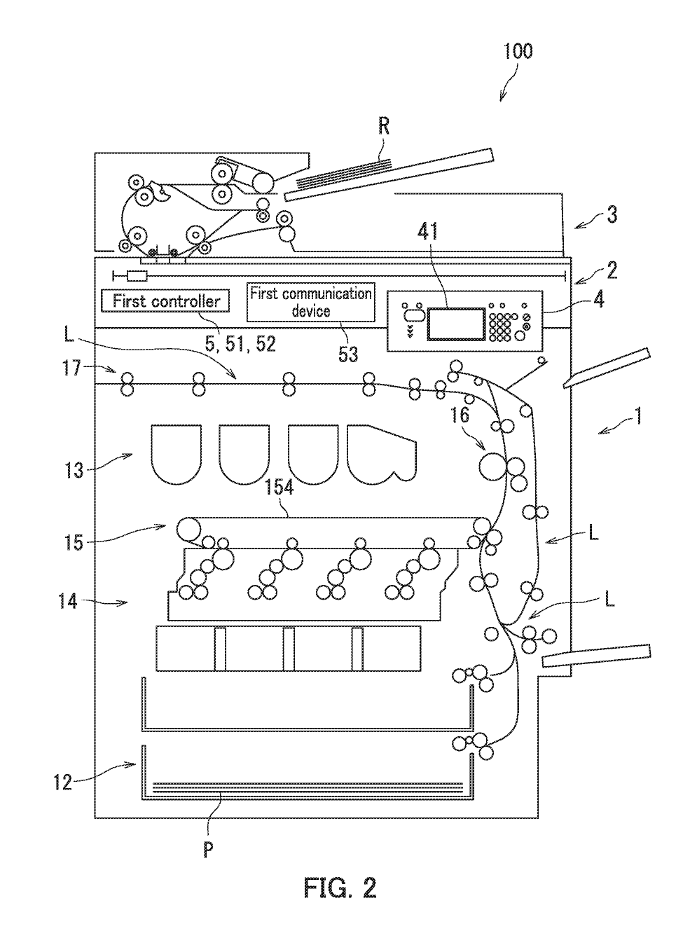

The following describes a configuration of the image forming apparatus 100 according to the embodiment of the present disclosure with reference to FIG. 2. FIG. 2 is a diagram illustrating the configuration of the image forming apparatus 100. The image forming apparatus 100 is a color multifunction peripheral. The image forming apparatus 100 reads an image from an original document R and forms an image on paper P using toner.

As illustrated in FIG. 2, the image forming apparatus 100 includes an image forming unit 1, an image reading unit 2, a document feeding unit 3, an operation panel 4, a first controller 5, and a first communication device 53. The image forming unit 1 forms the image on the paper P. The image reading unit 2 reads the image from the original document R and generates image information. The document feeding unit 3 feeds the original document R to the image reading unit 2. The operation panel 4 receives an operation from a user. The first controller 5 controls operation of the image forming apparatus 100. The first communication device 53 performs short-range wireless communication.

The image forming unit 1 includes a paper feeder 12, a conveyance device L, a toner supply device 13, an image forming device 14, a fixing device 16, and an ejection device 17. The image forming device 14 includes a transfer device 15.

The paper feeder 12 feeds the paper P to the conveyance device L. The conveyance device L conveys the paper P to the ejection device 17 through the transfer device 15 and the fixing device 16. The paper P is equivalent to an example of what is referred to as a “recording medium”.

The toner supply device 13 supplies the toner to the image forming device 14. The image forming device 14 forms the image on the paper P.

The transfer device 15 includes an intermediate transfer belt 154. The image forming device 14 transfers cyan, magenta, yellow, and black toner images onto the intermediate transfer belt 154. The toner images of the respective colors are superimposed on the intermediate transfer belt 154, and thus an image is formed on the intermediate transfer belt 154. The transfer device 15 transfers the image formed on the intermediate transfer belt 154 onto the paper P. As a result, the image is formed on the paper P.

After the image is formed on the paper P, the fixing device 16 applies heat and pressure to the paper P to fix the image on the paper P. The ejection device 17 ejects the paper P out of the image forming apparatus 100.

The operation panel 4 includes a touch panel 41. The touch panel 41 for example includes a liquid crystal display (LCD) and displays various images. The touch panel 41 also includes a touch sensor and receives an operation from the user.

The first controller 5 includes a processor 51 and storage 52. The processor 51 for example includes a central processing unit (CPU). The storage 52 includes memory, such as semiconductor memory, and optionally includes a hard disk drive (HDD). The storage 52 stores therein a control program.

The following describes the configuration of the wearable terminal device 200 with reference to FIG. 3. FIG. 3 is a diagram illustrating the configuration of the wearable terminal device 200. As illustrated in FIG. 3, the wearable terminal device 200 includes a band 60, a second controller 6, a second communication device 63, a display 64, a vibratory motor 65, an acceleration sensor 66, a pressure sensor 67, and a pressurization mechanism 68.

The band 60 is stretchable and is to be wound around an arm of the user. The band 60 is capable of altering pressure to apply to the arm of the user. The band 60 includes a cuff (not illustrated).

The second controller 6 includes a processor 61 and storage 62. The processor 61 for example includes a central processing unit (CPU). The storage 62 includes memory, such as semiconductor memory, and optionally includes a hard disk drive (HDD). The storage 62 stores therein a control program. The processor 61 controls the wearable terminal device 200 by executing the control program.

The second communication device 63 performs short-range wireless communication.

The display 64 for example includes an LCD and displays various images.

The vibratory motor 65 produces a vibration. Specifically, a weight is eccentrically provided on a tip of a rotary shaft of the vibratory motor 65. Rotation of the vibratory motor 65 causes the centroid position of the weight to circle around the rotary shaft, thus producing a vibration.

The acceleration sensor 66 detects acceleration.

The pressure sensor 67 is disposed on an inner circumferential surface of the band 60 and detects pressure.

The pressurization mechanism 68 alters pressure to apply to the arm from the band 60. Specifically, the pressurization mechanism 68 increases the pressure to apply to the arm of the user by inflating the cuff and decreases the pressure to apply to the arm of the user by deflating the cuff.

The following describes a configuration of the first controller 5 and a configuration of the second controller 6 according to the embodiment of the present disclosure with reference to FIGS. 1 to 4. FIG. 4 is a diagram illustrating the configuration of the first controller 5 of the image forming apparatus 100 and the configuration of the second controller 6 of the wearable terminal device 200.

As illustrated in FIG. 4, the first controller 5 includes a communication control section 501, an acquisition section 502, a determination section 503, a generation section 504, a display section 505, an update section 506, a transmission section 507, and an instruction section 508. Specifically, the processor 51 functions as the communication control section 501, the acquisition section 502, the determination section 503, the generation section 504, the display section 505, the update section 506, the transmission section 507, and the instruction section 508 by executing the control program.

The communication control section 501 controls operation of the first communication device 53. Specifically, the communication control section 501 determines whether or not the first communication device 53 is ready for communication with the wearable terminal device 200 (the second communication device 63). The communication control section 501 is equivalent to a “part of a communication device”. The first communication device 53 and the communication control section 501 are equivalent to an example of what is referred to as a “communication device”.

The acquisition section 502 acquires the information indicating the target value HT from the server 500. The acquisition section 502 also acquires the information indicating the measurement value HM from the wearable terminal device 200 via the first communication device 53. Specifically, upon the communication control section 501 determining that the first communication device 53 is ready for communication with the second communication device 63, the acquisition section 502 acquires the information indicating the measurement value HM from the wearable terminal device 200 via the first communication device 53.

The determination section 503 determines a food ingredient FC recommended for the user to ingest based on the target value HT and the measurement value HM.

The generation section 504 generates a list LF of food recommended for the user to purchase based on the food ingredient FC.

The display section 505 displays the list LF on the touch panel 41.

The update section 506 updates the list LF based on an operation of the wearable terminal device 200.

The transmission section 507 transmits information indicating the list LF to the wearable terminal device 200.

The instruction section 508 instructs the image forming device 14 to form an image exhibiting the list LF on the paper P. In accordance with the instruction from the instruction section 508, the image forming device 14 forms the image exhibiting the list LF on the paper P.

According to the embodiment of the present disclosure, as described above with reference to FIGS. 1 to 4, the determination section 503 determines the food ingredient FC recommended for the user to ingest based on the target value HT and the measurement value HM. The generation section 504 generates the list LF of food recommended for the user to purchase based on the food ingredient FC. It is therefore possible to generate the list LF of food suitable for the health status of the user by frequently updating the measurement value HM.

According to the embodiment of the present disclosure, the acquisition section 502 acquires the information indicating the target value HT from the server 500, but the present disclosure is not limited to such an embodiment. The acquisition section 502 may acquire the information indicating the target value HT from any source. For example, the acquisition section 502 may acquire the information indicating the target value HT from the wearable terminal device 200.

Next, the configuration of the second controller 6 will be described with reference to FIG. 4. As illustrated in FIG. 4, the second controller 6 includes a communication control section 601, a determination section 602, a notification section 603, a detection section 604, a measurement control section 605, and a blood pressure storage section 621. Specifically, the processor 61 functions as the communication control section 601, the determination section 602, the notification section 603, the detection section 604, and the measurement control section 605 by executing the control program. The storage 62 functions as the blood pressure storage section 621.

The blood pressure storage section 621 stores therein the measurement value HM. Specifically, the blood pressure storage section 621 stores a measured blood pressure level in association with a date and time of measurement. The measured blood pressure level is equivalent to an example of the measurement value HM.

The communication control section 601 controls operation of the second communication device 63.

The determination section 602 receives, from the POS terminal device 400, the food information FJ indicating a type of food purchased by the user and determines whether or not the food indicated by the food information FJ is included in the list LF. The determination section 602 is equivalent to an example of what is referred to as a “first determination section”. The determination section 602 is also equivalent to an example of what is referred to as a “second determination section”.

Upon the determination section 602 determining that the food indicated by the food information FJ received from the fourth communication device 401 is not included in the list LF, the notification section 603 performs the following process. That is, the notification section 603 causes the vibratory motor 65 to produce a vibration and displays, on the display 64, information indicating that the food indicated by the food information FJ is not included in the list LF. The notification section 603 is equivalent to an example of what is referred to as a “second notification section”.

The detection section 604 detects movement of the user based on a result of the detection by the acceleration sensor 66.

The measurement control section 605 measures a blood pressure level of the user using the pressure sensor 67 and the pressurization mechanism 68, and stores the measured blood pressure level in the blood pressure storage section 621. The pressure sensor 67 and the pressurization mechanism 68 form an example of what is referred to as a “measurement device”.

According to the embodiment of the present disclosure, as described above with reference to FIGS. 1 to 4, the determination section 602 receives the food information FJ indicating the type of the food purchased by the user from the POS terminal device 400 and determines whether or not the food indicated by the food information FJ is included in the list LF of the recommended food. The notification section 603 notifies the user of a result of the determination by the determination section 602. The user can therefore immediately know whether or not the food purchased by the user is included in the list LF of the recommended food. Thus, user convenience is enhanced.

Upon the determination section 602 determining that the food indicated by the food information FJ from the fourth communication device 401 of the POS terminal device 400 is not included in the list LF, for example, the notification section 603 performs the following process. That is, the notification section 603 causes the vibratory motor 65 to produce a vibration and displays, on the display 64, information indicating that the food indicated by the food information FJ is not included in the list LF. The user can therefore immediately know that the food purchased by the user is not included in the list LF. Thus, user convenience is enhanced.

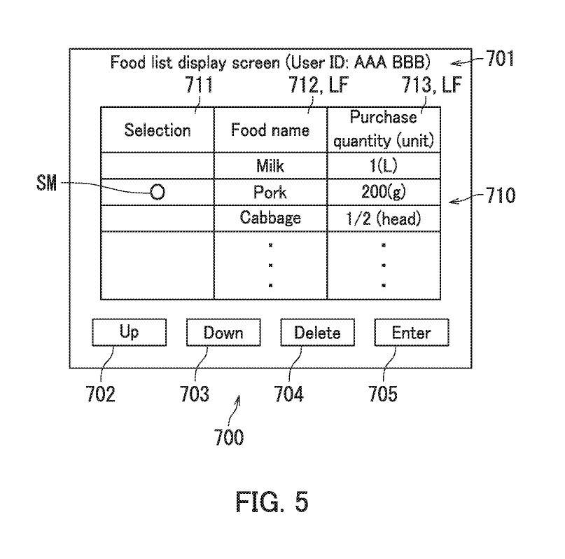

The following describes a food list display screen 700 with reference to FIGS. 2 to 5. FIG. 5 is a screen image illustrating an example of the food list display screen 700. The display section 505 displays the food list display screen 700 on the touch panel 41.

As illustrated in FIG. 5, the food list display screen 700 shows a user display section 701, a list display section 710, an up button 702, a down button 703, a delete button 704, and an enter button 705.

The user display section 701 displays identification information of the user. For example, the user display section 701 displays a user ID.

The list display section 710 displays the list LF. Specifically, the list display section 710 includes sections of selection 711, food name 712, and purchase quantity 713. The food name 712 and the purchase quantity 713 form the list LF.

The section of the selection 711 shows a position of a selected food in the list LF. For example, the section of the selection 711 displays a selection mark SM in a position corresponding to the selected food. The selection mark SM is for example an open circle.

The food name 712 means a name of food recommended for the user to purchase.

The purchase quantity 713 means a quantity of food recommended for the user to purchase.

The up button 702 is to be touched by the user in order to move the selection mark SM to an upper cell of the section of the selection 711. The update section 506 also moves the selection mark SM to an upper cell of the section of the selection 711 in response to the detection section 604 detecting a first movement of the user. The first movement is for example to move the wearable terminal device 200 upward.

Specifically, the acceleration sensor 66 detects upward acceleration when the wearable terminal device 200 is moved upward. The detection section 604 detects the first movement of the user through the acceleration sensor 66 detecting the upward acceleration. Accordingly, information indicating that the detection section 604 has detected the first movement is transmitted to the first controller 5 via the second communication device 63 and the first communication device 53. In response to the transmission of the information indicating that the detection section 604 has detected the first movement, the update section 506 moves the selection mark SM to an upper cell of the section of the selection 711.

The down button 703 is to be touched by the user in order to move the selection mark SM to a lower cell of the section of the selection 711. The update section 506 also moves the selection mark SM to a lower cell of the section of the selection 711 in response to the detection section 604 detecting a second movement of the user. The second movement is for example to move the wearable terminal device 200 downward.

Specifically, the acceleration sensor 66 detects downward acceleration when the wearable terminal device 200 is moved downward. The detection section 604 detects the second movement of the user through the acceleration sensor 66 detecting the downward acceleration. Accordingly, information indicating that the detection section 604 has detected the second movement is transmitted to the first controller 5 via the second communication device 63 and the first communication device 53. In response to the transmission of the information indicating that the detection section 604 has detected the second movement, the update section 506 moves the selection mark SM to a lower cell of the section of the selection 711.

The delete button 704 is to be touched by the user in order to delete food for which the selection mark SM is being displayed. The update section 506 also deletes food for which the selection mark SM is being displayed and updates the list LF in response to the detection section 604 detecting a third movement of the user. The third movement is for example to move the wearable terminal device 200 rightward.

Specifically, the acceleration sensor 66 detects rightward acceleration when the wearable terminal device 200 is moved rightward. The detection section 604 detects the third movement of the user through the acceleration sensor 66 detecting the rightward acceleration. Accordingly, information indicating that the detection section 604 has detected the third movement is transmitted to the first controller 5 via the second communication device 63 and the first communication device 53. The update section 506 deletes the food for which the selection mark SM is being displayed and updates the list LF in response to the transmission of the information indicating that the detection section 604 has detected the third movement.

The enter button 705 is to be touched by the user in order to confirm a list displayed on the list display section 710 as the list LF of the food recommended for the user to purchase. The update section 506 also updates the list LF in response to the detection section 604 detecting a fourth movement of the user. The fourth movement is for example to move the wearable terminal device 200 leftward.

Specifically, the acceleration sensor 66 detects leftward acceleration when the wearable terminal device 200 is moved leftward. The detection section 604 detects the fourth movement of the user through the acceleration sensor 66 detecting the leftward acceleration. Accordingly, information indicating that the detection section 604 has detected the fourth movement is transmitted to the first controller 5 via the second communication device 63 and the first communication device 53. The update section 506 updates the list LF in response to the transmission the information indicating that the detection section 604 has detected the fourth movement.

Once the update section 506 has updated the list LF, the transmission section 507 transmits the list LF to the wearable terminal device 200.

According to the embodiment of the present disclosure, as described above with reference to FIGS. 2 to 5, the display section 505 displays the list LF of the food recommended for the user to purchase on the touch panel 41 as soon as the first communication device 53 and the second communication device 63 are ready for communication with each other. The user can therefore immediately check the list LF of the food recommended to purchase. Thus, user convenience is enhanced.

The update section 506 updates the list LF based on an operation of the wearable terminal device 200. It is therefore possible to update the list LF without operating the operation panel 4. Thus, user convenience is enhanced.

Furthermore, the transmission section 507 transmits the information indicating the list LF to the wearable terminal device 200. The user can therefore check the list LF on the wearable terminal device 200. For example, the user can check the list LF by displaying the list LF on the display 64. Thus, user convenience is enhanced.

The following describes a process performed by the second controller 6 of the wearable terminal device 200, and the third communication device 301 and the third storage 302 on the grocery shelving unit 300 with reference to FIGS. 2 to 6. FIG. 6 is a diagram illustrating an example of the grocery shelving unit 300.

As illustrated in FIG. 6, a user US is wearing the wearable terminal device 200 on an arm.

The grocery shelving unit 300 includes three shelves, among which a top shelf is provided with a third communication device 301a and third storage 302a. First food FD1 is placed on the top shelf. The food information FJ indicating the first food FD1 is stored in the third storage 302a. The third communication device 301a transmits the food information FJ indicating the first food FD1 to the wearable terminal device 200 once the third communication device 301a is ready for communication with the wearable terminal device 200.

A middle shelf of the grocery shelving unit 300 is provided with a third communication device 301b and third storage 302b. Second food FD2 is placed on the middle shelf. The food information FJ indicating the second food FD2 is stored in the third storage 302b. The third communication device 301b transmits the food information FJ indicating the second food FD2 to the wearable terminal device 200 once the third communication device 301b is ready for communication with the wearable terminal device 200.

A lower shelf of the grocery shelving unit 300 is provided with a third communication device 301c and third storage 302c. Third food FD3 is placed on the lower shelf. The food information FJ indicating the third food FD3 is stored in the third storage 302c. The third communication device 301c transmits the food information FJ indicating the third food FD3 to the wearable terminal device 200 once the third communication device 301c is ready for communication with the wearable terminal device 200.

The determination section 602 of the second controller 6 of the wearable terminal device 200 performs the following process. That is, the determination section 602 receives the food information FJ from the third communication device 301 on the grocery shelving unit 300 and determines whether or not the food indicated by the food information FJ is included in the list LF. Specifically, the determination section 602 receives the food information FJ indicating the first food FD1 from the third communication device 301a, receives the food information FJ indicating the second food FD2 from the third communication device 301b, and receives the food information FJ indicating the third food FD3 from the third communication device 301c.

The notification section 603 notifies the user US of a result of the determination by the determination section 602. Specifically, upon the determination section 602 determining that the food indicated by the food information FJ from the third communication device 301 on the grocery shelving unit 300 is included in the list LF, the notification section 603 performs the following process. That is, the notification section 603 notifies the user US of a result of the determination by the determination section 602. Specifically, the notification section 603 causes the vibratory motor 65 to produce a vibration and displays, on the display 64, information indicating that the food indicated by the food information FJ is included in the list LF.

According to the embodiment of the present disclosure, as described above with reference to FIGS. 2 to 6, the determination section 602 receives the food information FJ from the third communication device 301 on the grocery shelving unit 300 and determines whether or not the food indicated by the food information FJ is included in the list LF. Furthermore, the notification section 603 notifies the user US of a result of the determination by the determination section 602. The user US can therefore immediately know whether or not the food indicated by the food information FJ is included in the list LF. Thus, convenience of the user US is enhanced.

Since the notification section 603 causes the vibratory motor 65 to produce a vibration upon the determination section 602 determining that the food indicated by the food information FJ from the third communication device 301 is included in the list LF, the user US need not see the display 64 of the wearable terminal device 200. The user US can therefore reliably know that the food indicated by the food information FJ is included in the list LF. Thus, convenience of the user US is enhanced.

Furthermore, upon the determination section 602 determining that the food indicated by the food information FJ from the third communication device 301 is included in the list LF, the notification section 603 displays, on the display 64, information indicating that the food indicated by the food information FJ is included in the list LF. The user US can therefore check the food information FJ of the food determined to be included in the list LF.

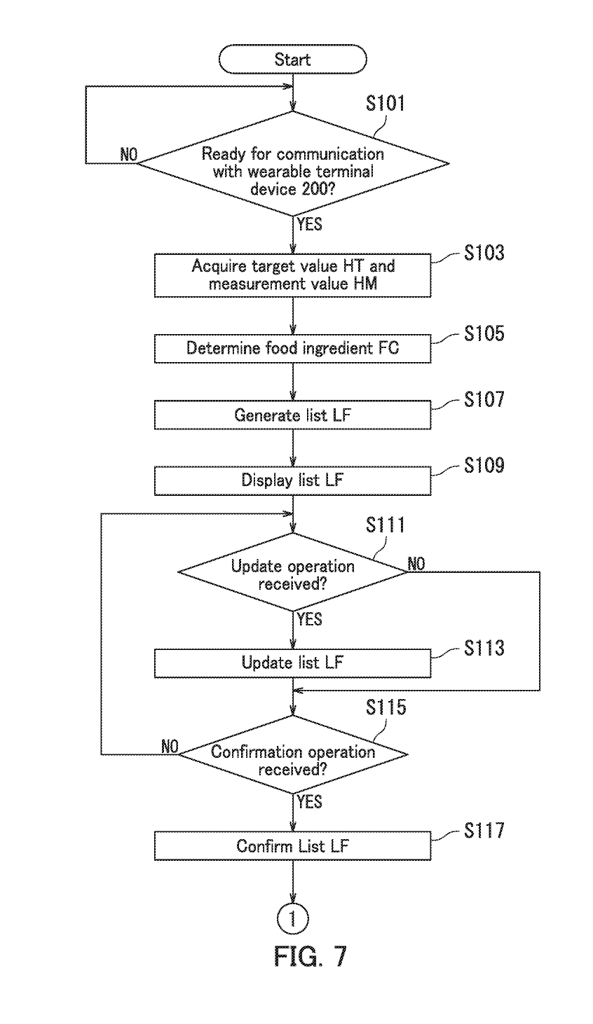

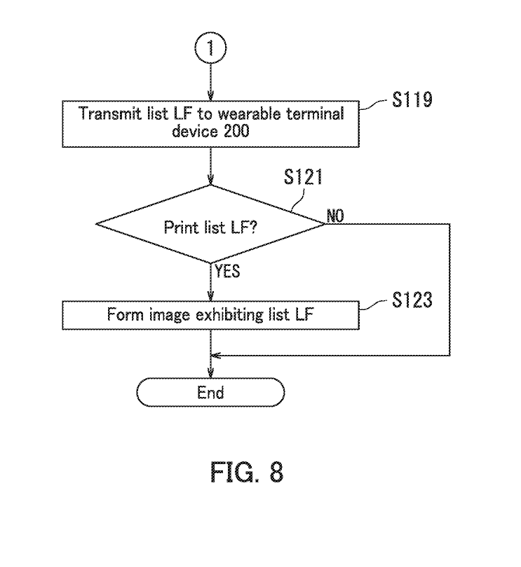

The following describes a process performed by the first controller 5 in the image forming apparatus 100 with reference to FIGS. 2 to 8. FIGS. 7 and 8 are flowcharts illustrating an example of the process performed by the first controller 5.

First, in Step S101 as illustrated in FIG. 7, the communication control section 501 determines whether or not the first communication device 53 is ready for communication with the wearable terminal device 200 (the second communication device 63).

Upon the communication control section 501 determining that the first communication device 53 is not ready for communication with the wearable terminal device 200 (NO in step S101), the process is put into a standby state. Upon the communication control section 501 determining that the first communication device 53 is ready for communication with the wearable terminal device 200 (YES in step S101), the process advances to step S103.

Next, in step S103, the acquisition section 502 acquires the measurement value HM from the wearable terminal device 200 and acquires the target value HT from the server 500.

Next, in step S105, the determination section 503 determines the food ingredient FC recommended for the user to ingest based on the target value HT and the measurement value HM.

Next, in step S107, the generation section 504 generates the list LF of food recommended for the user to purchase based on the food ingredient FC.

Next, in step S109, the display section 505 displays the list LF on the touch panel 41.

Next, in step S111, the update section 506 determines whether or not an update operation has been received from the wearable terminal device 200 or the touch panel 41. The update operation is an operation to update the list LF.

Upon the update section 506 determining that the update operation has not been received (NO in step S111), the process advances to step S115. Upon the update section 506 determining that the update operation has been received (YES in step S111), the process advances to step S113.

Next, in step S113, the update section 506 updates the list LF in accordance with the update operation.

Next, in step S115, the update section 506 determines whether or not a confirmation operation has been received from the wearable terminal device 200 or the touch panel 41. The confirmation operation is an operation to confirm the list LF.

Upon the update section 506 determining that the confirmation operation has not been received (NO in step S115), the process returns to step S111. Upon the update section 506 determining that the confirmation operation has been received (YES in step S115), the process advances to step S117.

Next, in step S117, the update section 506 confirms the list LF.

Next, in step S119 as illustrated in FIG. 8, the transmission section 507 transmits information indicating the list LF to the wearable terminal device 200.

Next, in step S121, the instruction section 508 determines whether or not to print the list LF.

Upon the instruction section 508 determining not to print the list LF (NO in step S121), the process ends. Upon the instruction section 508 determining to print the list LF (YES in step S121), the process advances to step S123.

Next, in step S123, the image forming device 14 forms an image exhibiting the list LF on the paper P in accordance with the instruction by the instruction section 508, and the process ends.

According to the embodiment of the present disclosure, as described above with reference to FIGS. 2 to 8, the image forming device 14 forms the image exhibiting the list LF on the paper P. The user can therefore readily check the list LF. Thus, user convenience is enhanced.

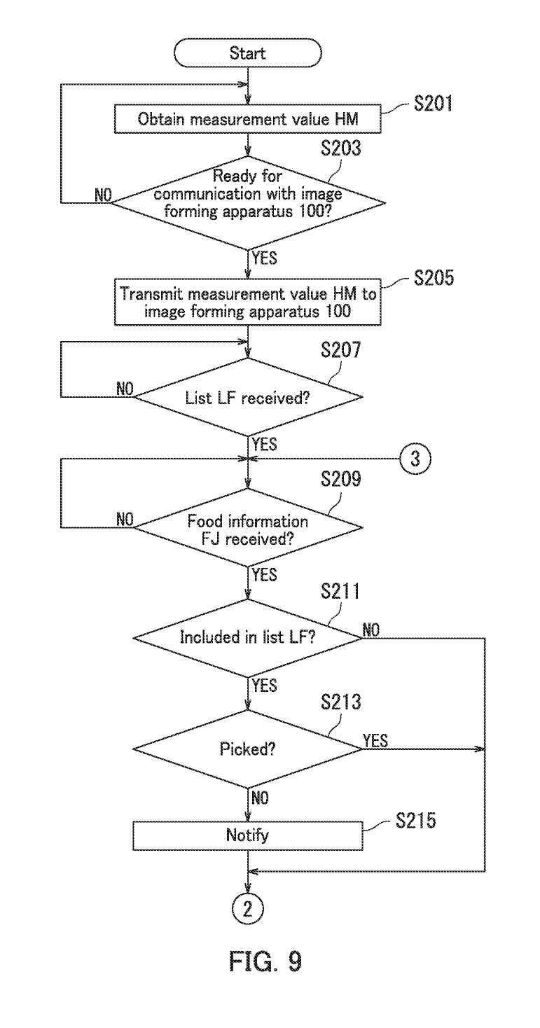

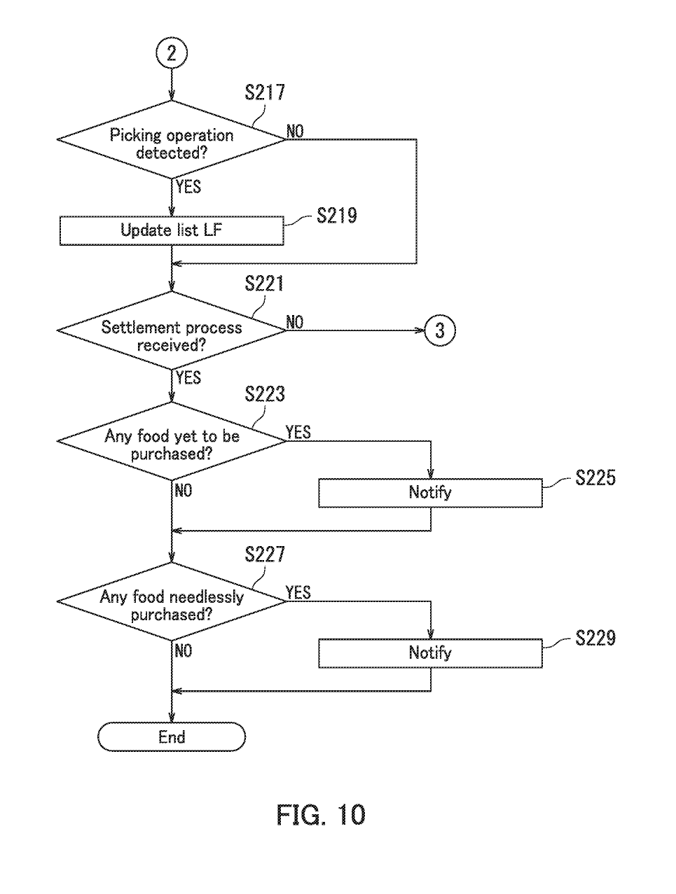

The following describes a process performed by the second controller 6 in the wearable terminal device 200 with reference to FIGS. 2 to 6, 9, and 10. FIGS. 9 and 10 are flowcharts illustrating an example of the process performed by the second controller 6.

First, in step S201 as illustrated in FIG. 9, the measurement control section 605 performs measurement to obtain the measurement value HM. Specifically, the measurement control section 605 measures a blood pressure level of the user using the pressure sensor 67 and the pressurization mechanism 68.

Next, in step S203, the communication control section 601 determines whether or not the wearable terminal device 200 is ready for communication with the image forming apparatus 100. Specifically, the communication control section 601 determines whether or not the second communication device 63 is ready for communication with the first communication device 53.

Upon the communication control section 601 determining that the wearable terminal device 200 is not ready for communication with the image forming apparatus 100 (NO in step S203), the process returns to step S201. Upon the communication control section 601 determining that the wearable terminal device 200 is ready for communication with the image forming apparatus 100 (YES in step S203), the process advances to step S205.

Next, in step S205, the communication control section 601 transmits the information indicating the measurement value HM to the image forming apparatus 100.

Next, in step S207, the communication control section 601 determines whether or not the information indicating the list LF has been received from the image forming apparatus 100.

Upon the communication control section 601 determining that the information indicating the list LF has not been received from the image forming apparatus 100 (NO in step S207), the process is put into a standby state. Upon the communication control section 601 determining that the information indicating the list LF has been received from the image forming apparatus 100 (YES in step S207), the process advances to step S209.

Next, in step S209, the communication control section 601 determines whether or not the food information FJ has been received from the third communication device 301 on the grocery shelving unit 300.

Upon the communication control section 601 determining that the food information FJ has not been received (NO in step S209), the process is put into a standby state. Upon the communication control section 601 determining that the food information FJ has been received (YES in step S209), the process advances to step S211.

Next, in step S211, the determination section 602 determines whether or not the food indicated by the food information FJ is included in the list LF.

Upon the determination section 602 determining that the food indicated by the food information FJ is not included in the list LF (NO in step S211), the process advances to step S217 in FIG. 10. Upon the determination section 602 determining that the food indicated by the food information FJ is included in the list LF (YES in step S211), the process advances to step S213.

Next, in step S213, the determination section 602 determines whether or not the food indicated by the food information FJ has been picked. “Food being picked” means that food FD has been picked from the grocery shelving unit 300.

Upon the determination section 602 determining that the food indicated by the food information FJ has been picked (YES in step S213), the process advances to step S217 in FIG. 10. Upon the determination section 602 determining that the food indicated by the food information FJ has not been picked (NO in step S213), the process advances to step S215.

Next, in step S215, the notification section 603 notifies the user of a result of the determination by the determination section 602. Specifically, the notification section 603 causes the vibratory motor 65 to produce a vibration and displays, on the display 64, information indicating that the food indicated by the food information FJ is included in the list LF.

Next, in step S217 as illustrated in FIG. 10, the detection section 604 determines whether or not a picking operation has been detected. The “picking operation” means an operation to pick the food FD from the grocery shelving unit 300.

Upon the detection section 604 determining that the picking operation has not been detected (NO in step S217), the process advances to step S221. Upon the detection section 604 determining that the picking operation has been detected (YES in step S217), the process advances to step S219.

Next, in step S219, the second controller 6 updates the list LF. Specifically, the second controller 6 adds the information indicating that the food FD has been picked to the list LF. The food FD means the food for which the picking operation has been detected by the detection section 604.

Next, in step S221, the second controller 6 determines whether or not a settlement process performed by the POS terminal device 400 has been received. Specifically, the second controller 6 determines that the settlement process performed by the POS terminal device 400 has been received when the wearable terminal device 200 is ready for communication with the POS terminal device 400 and receives the food information FJ indicating the purchased food FD from the POS terminal device 400.

Upon the second controller 6 determining that the settlement process performed by the POS terminal device 400 has not been received (NO in step S221), the process returns to step S209 in FIG. 9. Upon the second controller 6 determining that the settlement process performed by the POS terminal device 400 has been received (YES in step S221), the process advances to step S223.

Next, in step S223, the determination section 602 determines whether or not there is any food yet to be purchased. “Food yet to be purchased” means food that is included in the list LF but is not included in the food purchased. That is, “food yet to be purchased” means food FD recommended for the user to purchase but not yet purchased by the user.

Upon the determination section 602 determining that there is no food yet to be purchased (NO in step S223), the process advances to step S227. Upon the determination section 602 determining that there is food yet to be purchased (YES in step S223), the process advances to step S225.

Next, in step S225, the notification section 603 notifies the user that there is food yet to be purchased. Specifically, the notification section 603 causes the vibratory motor 65 to produce a vibration and displays, on the display 64, information indicating that there is food yet to be purchased.

Next, in step S227, the determination section 602 determines whether or not there is any food needlessly purchased. “Food needlessly purchased” means food that has been purchased even though the food is not included in the list LF. That is, “food needlessly purchased” means food that has been purchased by the user but that should not have been purchased by the user.

Upon the determination section 602 determining that there is no food needlessly purchased (NO in step S227), the process ends. Upon the determination section 602 determining that there is food needlessly purchased (YES in step S227), the process advances to step S229.

Next, in step S229, the notification section 603 notifies the user that there is food needlessly purchased. Specifically, the notification section 603 causes the vibratory motor 65 to produce a vibration and displays, on the display 64, information indicating that there is food needlessly purchased. Thereafter, the process ends.

According to the embodiment of the present disclosure, as described above with reference to FIGS. 2 to 6, 9, and 10, the wearable terminal device 200 performs measurement to obtain the measurement value HM. It is therefore possible to frequently update the measurement value HM. Accordingly, it is possible to reliably generate the list LF suitable for the latest health status of the user.

The wearable terminal device 200 measures a blood pressure level of the user. It is therefore possible to frequently update the measurement value for blood pressure. Accordingly, it is possible to generate the list LF suitable for the latest blood pressure of the user.

The notification section 603 notifies the user that there is food yet to be purchased upon the determination section 602 determining that there is food yet to be purchased. The user can therefore readily know that there is food yet to be purchased. Thus, user convenience is enhanced.

The notification section 603 also notifies the user that there is food needlessly purchased upon the determination section 602 determining that there is food needlessly purchased. The user can therefore readily know that there is food needlessly purchased. Thus, user convenience is enhanced.

Through the above, an embodiment of the present disclosure has been described with reference to the drawings. However, the present disclosure is not limited to the above embodiment and may be implemented in various different forms that do not deviate from the essence of the present disclosure (for example, as described below in (1) to (3)). The drawings schematically illustrate elements of configuration in order to facilitate understanding and properties of elements of configuration illustrated in the drawings, such as thickness, length, and number thereof, may differ from actual properties thereof in order to facilitate preparation of the drawings. Furthermore, properties of elements of configuration described in the above embodiment, such as shape and dimensions, are merely examples and are not intended as specific limitations. Various alterations may be made so long as there is no substantial deviation from the configuration of the present disclosure.

(1) According to the embodiment of the present disclosure, as described above with reference to FIG. 3, the wearable terminal device 200 is for the user to wear on an arm. However, the present disclosure is not limited to such an embodiment. The wearable terminal device may be in any form so long as the user can wear the wearable terminal device on a body thereof. For example, the wearable terminal device may be for the user to wear on a wrist. For another example, the wearable terminal device may be for the user to wear on a head.

(2) According to the embodiment of the present disclosure, as described above with reference to FIG. 3, the wearable terminal device 200 measures a blood pressure level of the user. However, the present disclosure is not limited to such an embodiment. The wearable terminal device may measure any indicator so long as the wearable terminal device obtains the measurement value HM. For example, the wearable terminal device may measure heart rate. For another example, the wearable terminal device may measure a blood sugar level.

(3) According to the embodiment of the present disclosure, as described above with reference to FIG. 6, the third communication device 301 and the third storage 302 are provided on the grocery shelving unit 300. However, the present disclosure is not limited to such an embodiment. The wearable terminal device 200 may acquire the food information FJ of the food placed on the grocery shelving unit 300 by any means. An IC tag may be attached to the food, and the wearable terminal device 200 may acquire the food information FJ from the IC tag.

What is claimed is:

1. A food purchase supporting system comprising:
a wearable terminal device for a user to wear on a body thereof; and
an image forming apparatus communicatively connected to the wearable terminal device, wherein
the wearable terminal device stores a measurement value with respect to an indicator for management of health of the user, and
the image forming apparatus includes:
a communication device that communicates with the wearable terminal device;
an acquisition section configured to acquire the measurement value from the wearable terminal device via the communication device;
a determination section configured to determine a food ingredient recommended for the user to ingest based on the measurement value and a target value with respect to the indicator; and
a generation section configured to generate a list of food recommended for the user to purchase based on the food ingredient.

2. The food purchase supporting system according to claim 1, wherein
the image forming apparatus further includes:
an operation panel having a display; and
a display section configured to display the list on the display.

3. The food purchase supporting system according to claim 2, wherein
the image forming apparatus further includes an update section configured to update the list, and
the update section updates the list based on an operation of the wearable terminal device.

4. The food purchase supporting system according to claim 3, wherein
the wearable terminal device includes:
an acceleration sensor; and
a detection section configured to detect movement of the user based on a result of detection by the acceleration sensor, and
the update section updates the list based on a result of detection by the detection section.

5. The food purchase supporting system according to claim 1, wherein
the image forming apparatus further includes a transmission section configured to transmit information indicating the list to the wearable terminal device.

6. The food purchase supporting system according to claim 5, further comprising:
a communication device that communicates with the wearable terminal device and provided on a grocery shelving unit on which food is placed; and
storage that stores therein food information indicating a type of food placed on the grocery shelving unit, wherein
the wearable terminal device includes:
a first determination section configured to receive the food information and determine whether or not the food indicated by the food information is included in the list; and
a first notification section configured to notify the user of a result of determination by the first determination section.

7. The food purchase supporting system according to claim 5, further comprising
a point of sale (POS) terminal device communicatively connected to the wearable terminal device, wherein
the POS terminal device transmits food information indicating a type of food purchased by the user, and
the wearable terminal device includes:
a second determination section configured to receive the food information and determine whether or not the food indicated by the food information is included in the list; and
a second notification section configured to notify the user of a result of determination by the second determination section.

8. The food purchase supporting system according to claim 1, wherein
the wearable terminal device includes a measurement device that performs measurement to obtain the measurement value.

9. The food purchase supporting system according to claim 8, wherein
the measurement device measures blood pressure of the user.

10. An image forming apparatus for communicative connection to a wearable terminal device for a user to wear on a body thereof, the image forming apparatus comprising:
a communication device that communicates with the wearable terminal device;
an acquisition section configured to acquire a measurement value with respect to an indicator for management of health of the user from the wearable terminal device;
a determination section configured to determine a food ingredient recommended for the user to ingest based on the measurement value and a target value with respect to the indicator; and
a generation section configured to generate a list of food recommended for the user to purchase based on the food ingredient.

11. The image forming apparatus according to claim 10, further comprising
an image forming device that forms an image on a recording medium, wherein
the image forming device forms an image exhibiting the list on the recording medium.
